(19) United States
(12) Patent Application Publication
(10) Pub. No.: US 2024/0307364 A1
(43) Pub. Date: Sep. 19, 2024

(54) GAS TRANSMISSION EMULSION FOR INTRAVENOUS ADMINISTRATION

(71) Applicants: Elena Valentinovna ARSHINTSEVA, Republic of Mordovia, Saransk (RU); Sergei Yurevich PUSHKIN, Noginsk-9, Moscow region (RU)

(72) Inventors: Elena Valentinovna ARSHINTSEVA, Republic of Mordovia, Saransk (RU); Sergei Yurevich PUSHKIN, Noginsk-9, Moscow region (RU)

(21) Appl. No.: 18/576,118

(22) PCT Filed: Jul. 15, 2021
(86) PCT No.: PCT/RU2021/000301

§ 371 Date: Jan. 3, 2024

(51) Int. Cl. A61K 31/451; A61K 9/00; A61K 9/107; A61K 47/02; A61K 47/10

(52) U.S. Cl. CPC A61K 31/451; A61K 9/0019; A61K 9/107; A61K 47/02; A61K 47/10

(57) ABSTRACT

The invention relates to the field of medicine and veterinary science, in particular to a perfluororganic compound-based gas transmission emulsion. A gas transmission emulsion that comprises a perfluororganic compound, a poloxamer and at least one additional excipient, wherein it comprises perfluoromethylcyclohexylpiperidine washed off of alcohol-soluble and water-soluble organic compounds as a perfluororganic compound and comprises poloxamer-188 as a poloxamer and is intended for intravenous administration to the subject. Thus, a PFOC-based gas transmission emulsion is created, the qualitative composition of which ensures the achievement of the technical result consisting in substantially reducing reactogenicity of the emulsion and making it possible to administer it to the subject.

The invention relates to the field of medicine and veterinary science, in particular to a perfluororganic compound-based gas transmission emulsion.

Perfluororganic compounds-based gas transmission emulsions (PFOCGTE) used in medicine and veterinary are polyfunctional medicinal products capable of transferring blood gases (O₂, CO₂, NO), which consist of at least one perfluororganic compound (PFOC), wherein all hydrogen atoms are substituted with fluorine atoms, a surface-active substance (SAS) and at least one excipient that can be represented by salts solutions or thickening agents. Here, PFOC and SAS are the major constituents of emulsion.

Visually, PFOC are odourless clear colorless liquids, which are approximately 2 times as heavy as water. An exceptional C-F bond strength (485.6 KJ/mole) results in intermolecular forces of these compounds being very weak. Weak intermolecular forces in PFOC are exhibited in their extremely high capacity to dissolve gases, including blood gases. Thanks to the C-F bond strength, PFOC are characterized by chemical non-reactivity. They poorly dissolve in water and do not undergo metabolism in an organism. It should be noted that PFOC intravenously administered in the form of emulsions are retained in organs and tissues, retention time in the organs depends on the nature of said compounds and the dose of the administered emulsion. Study of biological properties of PFOC of different classes has shown that the clearance rate from organs depends on a number of interrelated physical-chemical parameters of these compounds: structure and molecular weight, boiling temperature and vapor tension, more particularly on critical temperature of dissolution of PFOC in hexane (T_{crH}). The values of the physical-chemical parameters used as a criterion for choosing PFOC for medical and veterinary purposes are given in Table 1.

TABLE 1
Values of physical-chemical parameters of PFOC

| | | | | | Grade of |
|---|---|---|---|---|---|
| | | T_{cr}H, | P, mm Hg | t_{1/2}, | clearance |
| Type of PFOC | MW | ° C. | (37°) | days | rate |
| Perfluorooctyl bromide (PFOB) | 493 | −20 | 10.5 | 4 | Fast |
| Perfluorobicyclo[4.3.0]nonane | 412 | 13 | 33 | 4 | Fast |
| Perfluorodecalin (PFD) | 462 | 22 | 12 | 7 | Fast |
| Perfluorodecahydroacenaphthene | 524 | 24 | 2 | 7 | Fast |
| Perfluorodimethyladamantane | 524 | 32 | n.d. | 10-20 | Fast |
| (PFDMA) | | | | | |
| Perfluoro-1-propyl-2 | 483 | 35 | 19 | 24 | Fast |
| methylpiperidine | | | | | |
| Perfluorotripropylamine (PFTPA) | 521 | 43 | 17 | 65 | Slow |
| Perfluoromethylcyclohexylpiperidine | 595 | 40 | 1 | 90 | Slow |
| (PFMCP) | | | | | |
| Perfluorotributylamine (PFTBA) | 671 | 61 | 1 | 400-900 | Slow |
| Perfluorodihexyl ether | 652 | 59 | 2 | 500 | Slow |

Note:
MW stands for molecular weight, T_{cr}H stands for a temperature at which equal volumes of PFOC and hexane are mixed together, P, mm Hg (37°) is a value of vapor tension at 37° C., t_{1/2} stands for elimination half-life.

It should be noted that T_{crH} is considered as a measure of relative solubility of PFOC in lipids, which characterizes the rate of their penetration through membranes. It is seen from the given data that the lower T_{crH} is, the smaller MW is and the higher vapor tension is, the better PFOC is soluble in lipids and the easier and faster it is eliminated from an organism. In this case, it is seen from the data of Table 1 that a correlation between said parameters and the elimination half-life of PFOC is discernible.

As already indicated above, the second major constituent of gas transmission emulsion is SAS. Among a great number of SAS, just few of them meet the requirements for their use in medicinal products intended for intravenous administration: they are non-toxic nonionic high-molecular substances, in particular poloxamers, and natural phospholipids (of soy, egg yolk). Their amount in an emulsion is as minimal as possible and is only determined by a necessity of satisfactory emulsification and homogenization of PFOC. Minimization of the amount of SAS is also attributed to the fact that they have an effect on toxicity and reactogenicity of a gas transmission emulsion.

The average diameter of particles of said emulsions depends both on the properties of PFOC, and on a capacity of SAS used in a gas transmission emulsion as a stabilizing additive to reduce surface tension on PFOC-water interface. The lower the value of surface tension is, the easier the process of emulsification proceeds and the smaller is the average diameter of the particles in the emulsion obtained. Said emulsions with certain stabilizing additive should have a long shelf life, and storage conditions should be simple. An indicator of PFOCGTE being stored is a change (an increase) in an average diameter of the particles of an emulsion when stored.

Thus, the main parameters of PFOCGTE which determine their quality and perspectivity of use are as follows: 1) the average diameter of the particles of PFOCGTE; 2) the diameter distribution of the particles of the emulsion; 3) toxicity of the emulsion, or LD₅₀; 4) the oxygen capacity of the emulsion; 5) sterility of the emulsion; 6) storability of the emulsion; 7) toxicity of the stabilizing additive-SAS; 8) the properties of PFOC used in the emulsion, namely MW, T_{crH}, P, t_{1/2}, chemical purity of the compound; 9) reactogenicity of the emulsion. Comparison of these indicators allows to evaluate the obtained PFOCGTE and to determine the advantage of one gas transmission emulsion over another.

A variety of PFOCGTE are known. Most often, the product engineers include to the emulsions the mixtures of PFOC that consist of at least one fast-eliminated PFOC, for example PFD or PFOB, and/or at least one slowly eliminated PFOC, for example PFTPA, PFMCP or PFTBA.

A high level of toxicity of the PFOCGTE consisting of a fast-eliminated PFOC only is noted by the researchers. For example, an emulsion that comprised only fast-eliminated PFD caused death of 100% of rabbits within 2-3 weeks following infusion at a dose of 10 ml/kg (Maevskii E. I. Vozmozhnye prichiny ostroi reaktogennosti emulsii perftoruglerodov, Vestnik IIF, p. 79-87, No. 1(39), 2016). To reduce toxicity and reactogenicity, a slowly eliminated PFOC is added to the fast-eliminated PFOC in an emulsion.

For example, medicinal product Fluosol DA 20% described in patent U.S. Pat. No. 4,252,827, comprising the relatively slowly eliminated PFTPA (t_{1/2}=65 days) in addition to the fast-eliminated PFD, is known from the art. Phospholipids of egg yolk and a nonionic high-molecular SAS, polyoxyethylene-polyoxypropylene copolymer with a molecular weight between 2000 and 20000, were used as a SAS in said medicinal product.

Said medicinal product was produced in the 1980s and 1990s by Green Cross Corporation (Japan) and had a number of considerable disadvantages: low efficiency, reactogenicity, which could be attributed to the oxidation of phospholipids of egg yolk and, as a result, to the formation of peroxy radicals in the emulsion, low stability of the composition when stored. Furthermore, Fluosol DA 20% could lead to vascular occlusion due to fairly coarse particles of an emulsion (up to 300 nm). It should also be noted that the preparation was technically difficult to use, because of its production was closed in 1995.

Also the medicinal product “Perftoran” described in patent RU2199311, which is an example of emulsion similar to Fluosol DA 20% and contains PFD and PFMCP (t_{1/2}=90 days) as a PFOC at the ratio of 2:1, is known from the art. Poloxamer Proxanol-268 (produced by NIOPIK, the RF) in an amount of 4 wt % is used in “Perftoran” emulsion as a SAS. It is noted by the researchers that during use of “Perftoran” emulsion by the patients reactogenicity varies between 2 and 20% and depends heavily on observing the conditions of storage, of defrosting, as well as on premedication of a patient. It should be noted that medicinal product “Perftoran” has received the marketing authorizations and is approved for clinical use in the following countries: the RF, Mexico, Ukraine, Kazakhstan and Uzbekistan.

Furthermore, the “Perfuzol” emulsion described in the article Perftoruglerodnye emulsii, stabilizirovannye neionogennymi bloksopolimerami/Vorobev S. I., Ivanitskii G. R., Makarov K. N. et al.//Perftoruglerodnye aktivnye sredy dlia meditsiny i biologii (novye aspekty issledovaniia). Sbornik nauchnykh trudov.—Pushchino, 1993, p. 33-46, is known from the art. Said emulsion comprises slowly eliminated PFOC PFMCP in an amount of 10 g, proxanol-168 (MW 5700 Da, POP<20%) in an amount of 3 g, hydroxyethylated starch (MW 80 kDa) in an amount of 3 g and salt composition.

The disadvantage of this gas transmission emulsion in terms of reactogenicity is the instability of said emulsion owing to it comprising low molecular Proxanol-168: in the event of a decrease in MW of a poloxamer its solubility increases, but its solubilizing capacity decreases. To create a stable and uniform surface layer on emulsion particles, it is recommended to use poloxamers with a molecular weight close to 9000 Da.

The closest prior art of the claimed invention is a gas transmission emulsion that comprises slowly eliminated PFOC PFMCP in a concentration between 1 and 10 wt %, Proxanol-268 (MW 13000 Da, POP 18-22%) in an amount of 3 wt % and electrolytes and is described in article Nizkokontsentrirovannye perftoruglerodnye krovezameshchaiushchie emulsii meditsinskogo naznacheniia/Vorobjev S. I., Kutushenko V. P., Bolevich S. B. et al.//Sbornik nauchnykh trudov. Netraditsionnye prirodnye resursy, innovatsionnye tekhnologii i produkty.—No. 21.—2013.—p. 112-123.

The advantage of said emulsion is good stability, a sufficient shelf life at a temperature of 20° C., since the bigger the POP block is in the poloxamer molecule, the stronger are its detergent properties and the better it emulsifies. (Vorobev S. I. Biologicheskie i fiziko-khimicheskie svoistva neionogennyh poverkhnostno-aktivnyh veshchestv//Rossiiskii bioterapevticheskii zhurnal.—No. 3, V. 8.—2009.—p. 3-8).

The disadvantage of this gas transmission emulsion in terms of toxicity and reactogenicity is that it comprises toxic organic and perfluoroorganic compounds present in PFMCP, which has not been washed off of said substances beforehand.

Said gas transmission emulsions, in which only PFMCP is contained as a PFOC, are intended for perfusion of isolated organs. These emulsions are not intended for intravenous administration.

The problem to be solved by this invention is to create a PFOC-based gas transmission emulsion, the qualitative composition of which will ensure the achievement of the technical result consisting in substantially reducing reactogenicity of the emulsion and in making it possible to administer it to the subject.

The problem posed is solved by developing a gas transmission emulsion that comprises a perfluororganic compound, a poloxamer and at least one additional excipient, wherein it comprises perfluoromethylcyclohexylpiperidine washed off of alcohol-soluble and water-soluble organic compounds as a perfluororganic compound and comprises poloxamer-188 as a poloxamer and is intended for intravenous administration to the subject.

As indicated above, emulsion quality is determined by the physical-chemical properties of a chosen PFOC and by the physical-chemical properties and the nature of a stabilizing additive. PFOC are not soluble in water and are themselves poor solvents for various water-soluble biologically active substances, therefore to be used as gas transmission media they are dispersed in a water solution of a stabilizing additive until the fine emulsions are obtained.

As a PFOC, the authors of the invention have chosen perfluorocarbon of generation II, PFMCP, in which the major constituent corresponds to formula I and which has no counterparts abroad. The presence of two cyclic structures in its molecule is favourable to the acceleration of its elimination from an organism, and the presence of a nitrogen heteroatom, aliphatic chains and CF₃ group facilitates the production of stable emulsions.

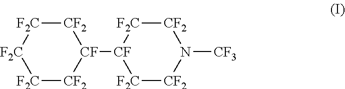

Said compound has the following physical-chemical characteristics: MW=595, T_{crH}=40° C., P=1 mm Hg (37°), t_{1/2}=90 days. It is a PFOC being slowly eliminated from an organism. Furthermore, the chemical purity of PFMCP, namely the absence in it of various kinds of impurities, that is, compounds having toxic or irritating properties, is an extremely important characteristic. Considering that in the prior art PFOCGTE comprised a PFMCP with a large number of impurities that caused reactogenicity of the emulsion, the authors of the present invention have removed said impurities having toxic or irritating properties from PFMCP. The impurities were removed from PFMCP by means of a 95% medical antiseptic solution. The efficiency of this method of purifying PFOC by washing off was controlled by chromatomass-spectrometric technique. Said data related to removal of impurities from PFMCP are given in the figures below.

A purpose behind using a stabilizing additive is to form an adsorption layer around the particles of PFOC, wherein the physical-chemical properties and the nature of the stabilizing additive determine the stability of the dispersion system and its reactogenicity. Here, the key parameters are the bond strength between SAS and the “core” of the particle of the emulsion, the character and density of arrangement of the molecules of SAS on its surface. Poloxamers are characterized by suitable values of these key parameters among SAS. They are block copolymers of polyoxyethylene and polyoxypropylene, which are used not only as excipients, but also as medicinal agents having useful biological properties. Poloxamer is most widely used as an emulsifier of fatty emulsions for intravenous administration, as well as a stabilizer. Furthermore, poloxamers are used in the treatment of pathological hydrophobic interactions in blood and other biological liquids, as they improve blood flow and reduce the adhesion of macromolecules and cells. It should be noted that there are several types of poloxamers, which differ from each other in MW, in the amount of polyoxyethylene (POE) and polyoxypropylene (POP) blocks in the molecule. In this case, according to the authors of the present invention, the use of poloxamers with a low and high MW in gas transmission emulsions, leads to the emergence of reactogenicity.

Considering the disadvantages of the prior art gas transmission emulsions, where both a mixture of poloxamer and egg yolk phospholipids, and poloxamers Proxanol-168 and Proxanol-268 (current name is “Emuxol-268” of grade “A”) were used as SAS, the authors of the present invention as a SAS have chosen a poloxamer with an optimal molecular weight and percentage of oxypropylene groups, which is poloxamer-188, produced by various manufacturers under the trademarks of Pluronic F68, Lutrol F68, Kolliphor P188 (BASF), Synperonic PE/F68 (Croda). In addition, poloxamer-188 is the closest in terms of its properties to Proxanol-168 and Proxanol-268 poloxamers, but differs substantially in terms of MW. The characteristics of said poloxamers are given in Table 2.

TABLE 2
Characteristics of the Proxanol-168, Proxanol-
268 and poloxamer-188 poloxamers

| | The classification | | |
|---|---|---|---|
| | of a poloxamer | | Number of |
| | according to USP | Molecular | oxypro- |
| Trade names of | (United States | weight, | pylene |
| poloxamers | Pharmacopoeia) | Da | blocks, % |
| Proxanol-168 | — | 8000¹, between | <20 |
| (produced by | | 5500 and 8500 | |
| NIOPIK), | | (the major | |
| Emuxol-168 | | fraction has a | |
| | | molecular weight | |
| | | of 6000 + 500)² | |
| Pluronic F68, Lutrol | Poloxamer 188 | 7680-9510³ | 16.3-20.1 |
| 68, Kolliphor 188 | | | |
| (produced by BASF), | | | |
| Synperonic PE/F68 | | | |
| (produced by | | | |
| CRODA) | | | |
| Proxanol-268 | — | 13000¹ | 18-22 |
| (produced by | | | |
| NIOPIK), Emuxol | | | |
| 268 of grade A | | | |

Note:
¹information accessed on the website of the manufacturer NIOPIK http://www.niopik.ru/products/oxyalkylation/;
²information from SU1451154, 1989, NIOPIK;
³information from United States Pharmacopoeia.

It should be further noted that according to the adopted name nomenclature for block copolymers digit 8 in the names proxanol-268, proxanol-168 and poloxamer-188 indicates that the average combined weight of the POE blocks in the molecule is 80%, and digits 26 in proxanol-268, digits 16 in proxanol-168 and digits 18 in poloxamer-188 denote that the average MW of the POP blocks, which constitute on average 20% of the weight of the molecule of proxanol-268, proxanol-168 and poloxamer-188, is 2600 Da, 1600 Da and 1800 Da respectively. Thus, the average MW of the block copolymer of proxanol-268, proxanol-168 and poloxamer-188 in total is 13000 Da, 8000 Da and 9000 Da respectively when the proportion of the blocks of POE and POP is on average 80% and 20%, which corresponds to data given in Table 2. Furthermore, thanks to using poloxamer-188, a stable gas transmission emulsion suitable for intravenous administration was obtained, given the fact that the average MW of poloxamer-188 is optimal and equals 9000 Da. It should also be noted that poloxamer-188 acts upon the cell membranes in a more “gentle” way thanks to a lower absolute value of the POP block (1800 Da) compared to proxanol-268 (2600 Da). (Vorobev S. I. Biologicheskie i fiziko-khimicheskie svoistva neionogennyh poverkhnostno-aktivnyh veshchestv//Rossiiskii bioterapevticheskii zhurnal.—No. 3, V. 8.—2009.—p. 3-8).

Thus, it can be concluded that neither Proxanol-168 (new name is Emuxol-168), nor Proxanol-268 (new name is Emuxol 268 of grade A) are poloxamer-188, although they are close to it in terms of some physical-chemical properties.

Preferably, the gas transmission emulsion is administered to a subject that is a mammal.

In this case, the mammal in a preferred embodiment is a human and “companion animals of human”. “Companion animals of human” means any domestic animal, including, but not limited to, cats, dogs, rabbits, guinea pigs, ferrets, hamsters, mice, horses, cows, goats, pigs. Preferred mammals are humans, dogs, cats and rabbits.

In a preferred embodiment, the additional excipient in a gas transmission emulsion is sodium chloride.

Also, in a preferred embodiment of the invention as claimed, the concentration of perfluoromethylcyclohexylpiperidine in a gas transmission emulsion is 1-20 wt %.

Preferably, the concentration of poloxamer-188 in a gas transmission emulsion is 2-8 wt %.

Preferably, the concentration of sodium chloride, when it is used as an excipient in a gas transmission emulsion, is 0.1-0.9 wt %.

One of the criteria for compliance with the technology in the production of a sterile intravenous medicinal product are the pyrogenicity analysis and the bacterial endotoxins analysis. The first analysis is carried out on rabbits, and the pyrogenicity criterion is the excess of the total temperature in the rabbits group, the second analysis determines the quantitative content of endotoxin in the culture medium samples. To assess reactogenicity, the authors conducted the above study, on the basis of which the reactogenicity test (RT) was later developed.

When comparing the results of the pyrogenicity analysis and the bacterial endotoxins analysis of the same production batches of “Perftoran” gas transmission emulsion, the authors found a significant discrepancy in the number of positive tests in the results of these two analyses. The standard pyrogenicity test was conducted on 3 rabbits. It was noted that on average for two out of five batches of “Perftoran” emulsion the pyrogenicity test had to be carried out anew due to the excess of the total temperature of the rabbits group or the excess of the maximum allowed temperature increase in one animal. In this case, the check of such batches for endotoxin content did not reveal a cause for temperature anomalies in rabbits. It was also noted that in at least 1-2 rabbits out of five in the repeated pyrogenicity test, the result showed a temperature increase close to the maximum allowed value or its excess. The authors of the invention made the assumption that these discrepancies are attributable not to pyrogenicity, but to adverse reactions in rabbits to administration of the gas transmission emulsion, which led to temperature increase, the adverse reactions being attributable to reactogenicity of “Perftoran” emulsion. Subsequently, the authors used this method to compare the compositions of the emulsions between each other according to the reactogenicity criterion (provisional name “reactogenicity test”, hereinafter to be referred to as RT).

The authors have subjected also an emulsion that is the closest to the present invention in terms of composition to a similar RT. The purpose of the research was to test the hypothesis of reduction in reactogenicity in the event of a change in the composition of the used PFOC.

The authors have prepared a PFO emulsion No. 1E containing in 100 ml of the emulsion: 10 g of PFMCP, 3 g of Proxanol-268, 0.6 g of NaCl, up to 100 ml of water for injections. RT of said emulsion was conducted on 5 rabbits. The results are given in Table 3.

TABLE 3
Results of reactogenicity test of gas transmission emulsion No. 1E.

| Number | Maximum allowable level of | Level of temperature |
|---|---|---|
| of rabbit | temperature rise, ° C. | rise, ° C. |
| 1 | 0.5 | 0.2 |
| 2 | 0.5 | 0.0 |
| 3 | 0.5 | 0.1 |
| 4 | 0.5 | 0.2 |
| 5 | 0.5 | 0.5 |

As a result of the conducted test, it was found that the obtained gas transmission emulsion is less reactogenic than “Perftoran” emulsion, but a sharp temperature change was observed in one rabbit.

Examples of gas transport emulsions according to the present invention, containing poloxamer-188 as a SAS and subjected to RT, are given below.

### EXAMPLE 1

Gas transmission emulsion No. 1 was prepared, which comprises PFMCP PFOC in a concentration of 1 wt %, Kolliphor P188 in a concentration of 2 wt %, NaCl in a concentration of 0.6 wt % and water. RT of said emulsion No. 1 was conducted on 5 rabbits. The results are given in Table 4.

TABLE 4
Results of reactogenicity test of gas transmission emulsion No. 1.

| Number | Maximum allowable level of | Level of temperature |
|---|---|---|
| of rabbit | temperature rise, ° C. | rise, ° C. |
| 1 | 0.5 | 0.1 |
| 2 | 0.5 | 0.2 |
| 3 | 0.5 | 0.0 |
| 4 | 0.5 | −0.2 |
| 5 | 0.5 | 0.1 |

As a result of the conducted test, it was found that the obtained gas transmission emulsion is not reactogenic.

### EXAMPLE 2

Gas transmission emulsion No. 2 was prepared, which comprises PFMCP PFOC in a concentration of 1 wt %, Kolliphor P188 in a concentration of 8 wt %, NaCl in a concentration of 0.6 wt % and water. RT of said emulsion No. 2 was conducted on 5 rabbits. The results are given in Table 5.

TABLE 5
Results of reactogenicity test of gas transmission emulsion No. 2.

| Number | Maximum allowable level of | Level of temperature |
|---|---|---|
| of rabbit | temperature rise, ° C. | rise, ° C. |
| 1 | 0.5 | 0.2 |
| 2 | 0.5 | 0.0 |
| 3 | 0.5 | 0.1 |
| 4 | 0.5 | 0.0 |
| 5 | 0.5 | −0.1 |

As a result of the conducted test, it was found that the obtained gas transmission emulsion is not reactogenic.

### EXAMPLE 3

Gas transmission emulsion No. 3 was prepared, which comprises PFMCP PFOC in a concentration of 20 wt %, Kolliphor P188 in a concentration of 4 wt %, NaCl in a concentration of 0.6 wt % and water. RT of said emulsion No. 3 was conducted on 5 rabbits. The results are given in Table 6.

TABLE 6
Results of reactogenicity test of gas transmission emulsion No. 3.

| Number | Maximum allowable level of | Level of temperature |
|---|---|---|
| of rabbit | temperature rise, ° C. | rise, ° C. |
| 1 | 0.5 | −0.2 |
| 2 | 0.5 | 0.3 |
| 3 | 0.5 | −0.1 |
| 4 | 0.5 | 0.0 |
| 5 | 0.5 | −0.1 |

As a result of the conducted test, it was found that the obtained gas transmission emulsion is not reactogenic.

### EXAMPLE 4

Gas transmission emulsion No. 4 was prepared, which comprises PFMCP PFOC in a concentration of 20 wt %, Kolliphor P188 in a concentration of 8 wt %, NaCl in a concentration of 0.6 wt % and water. RT of said emulsion No. 4 was conducted on 5 rabbits. The results are given in Table 7.

TABLE 7
Results of reactogenicity test of gas transmission emulsion No. 4.

| Number | Maximum allowable level of | Level of temperature |
|---|---|---|
| of rabbit | temperature rise, ° C. | rise, ° C. |
| 1 | 0.5 | 0.0 |
| 2 | 0.5 | 0.1 |
| 3 | 0.5 | −0.2 |
| 4 | 0.5 | −0.1 |
| 5 | 0.5 | 0.1 |

As a result of the conducted test, it was found that the obtained gas transmission emulsion is not reactogenic.

### EXAMPLE 5.

Gas transmission emulsion No. 5 was prepared, which comprises PFMCP PFOC in a concentration of 10 wt %, Kolliphor P188 in a concentration of 2 wt %, NaCl in a concentration of 0.6 wt % and water. RT of said emulsion No. 5 was conducted on 5 rabbits. The results are given in Table 8.

TABLE 8
Results of reactogenicity test of gas transmission emulsion No. 5.

| Number | Maximum allowable level of | Level of temperature |
|---|---|---|
| of rabbit | temperature rise, ° C. | rise, ° C. |
| 1 | 0.5 | 0.3 |
| 2 | 0.5 | 0.0 |
| 3 | 0.5 | −0.1 |
| 4 | 0.5 | 0.3 |
| 5 | 0.5 | 0.0 |

As a result of the conducted test, it was found that the obtained gas transmission emulsion is not reactogenic.

### EXAMPLE 6

Gas transmission emulsion No. 6 was prepared, which comprises PFMCP PFOC in a concentration of 5 wt %, Kolliphor P188 in a concentration of 4 wt %, NaCl in a concentration of 0.6 wt % and water. RT of said emulsion No. 6 was conducted on 5 rabbits. The results are given in Table 9.

TABLE 9
Results of reactogenicity test of gas transmission emulsion No. 6.

| Number | Maximum allowable level of | Level of temperature |
|---|---|---|
| of rabbit | temperature rise, ° C. | rise, ° C. |
| 1 | 0.5 | −0.1 |
| 2 | 0.5 | 0.2 |
| 3 | 0.5 | 0.1 |
| 4 | 0.5 | 0.2 |
| 5 | 0.5 | 0.0 |

As a result of the conducted test, it was found that the obtained gas transmission emulsion is not reactogenic.

All batches of gas transmission emulsions according to the present invention, which were prepared in various concentrations, showed a better reactogenicity test compared to both “Perftoran” emulsion and the prototype emulsion.

The present invention is also explained by means of the following drawings.

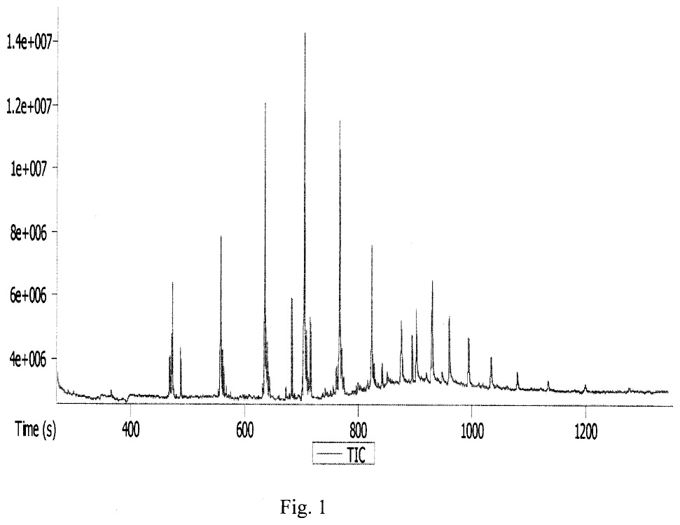

FIG. 1 is a chromatogram of a 95% antiseptic medical solution.

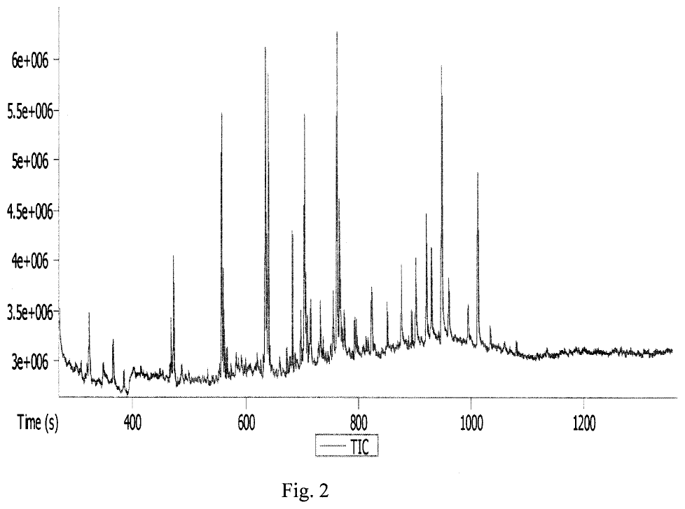

FIG. 2 is a chromatogram of a 95% antiseptic medical solution after removing impurities from PFMCP.

FIG. 1 is a chromatogram of a 95% antiseptic medical solution, which was a control sample. Said solution was analyzed for presence of compounds included in it on “Pegasus 4D” chromatomass spectrometer produced by LECO with electronic ionization and an RTX-5 MS capillary silicone column (30 m). Ionization energy—70 eV, temperature conditions: 50° C. (2 min)—20° C./min—300° C. (10 min), scanned masses 29-700 dalton.

FIG. 2 is a chromatogram of a 95% antiseptic medical solution after removing impurities from PFMCP. Said solution was the analyzed sample, once impurities were removed from the PFMCP. Alcohol-soluble and water-soluble organic compounds were determined on “Pegasus 4D” chromatomass spectrometer produced by LECO with electronic ionization and an RTX-5 MS capillary silicone column (30 m). Ionization energy—70 eV, temperature conditions: 50° C. (2 min)—20° C./min—300° C. (10 min), scanned masses 29-700 dalton. Computer libraries (NIST and WILEY) were used for qualitative determination. A 95% medical antiseptic solution was taken as a control sample.

The detected compounds from the analyzed sample are given in Table 10. It should be noted that where the concentrations of the detected compounds are approximately equal to the concentrations of the compounds in the control sample, said compounds were not included in Table 10. If their concentrations in the analyzed sample are statistically higher than in the control sample, they are highlighted in bold in Table 10. It should be noted that not all organic compounds have been identified. When calculating the concentration of alcohol-soluble and water-soluble perfluoroorganic and organic compounds with toxic and locally irritating properties, only substances identified by the chemical formula were taken into account. After washing PFMCP, the authors managed to purify said substance of impurities, namely toxic organic substances, the total concentration of which was 507.1 μg/l, which ultimately leads to a decrease in the reactogenicity of the PFMCP-based gas transmission emulsion.

TABLE 10
PFOC and organic compounds found in the 95% medical
antiseptic solution after washing PFMCP off

| | | Reten- | | Toxic- |
|---|---|---|---|---|
| No. of | | tion | | ity, |
| peak | Substance name | time | μg/l | yes/no |
| 1 | pentanol | 270.6 | 28 | yes |
| 2 | furfurol | 273.5 | 4.7 | yes |
| 3 | benzene, (1-ethoxyethyl)- | 293.2 | 10 | yes |
| 4 | cyclohexane, dodecafluoro- | 297.2 | 8.7 | yes |
| 6 | dimethylethoxyformamide | 305.1 | 16.3 | yes |
| 7 | perfluoroalkane | 305.6 | 5.3 | no |
| 8 | oxime-, methoxy-phenyl- | 307 | 20 | yes |
| 9 | perfluoro(methylcyclohexane) | 315.1 | 11 | no |
| 10 | O-ethyl-methoxy-phenyl-oxime | 324.2 | 43 | yes |
| 11 | perfluoromethylbutyl(4- | 328.5 | 53 | no |
| | methylcyclohexyl)amine | | | |
| 12 | phenylaminophenylcyclopropane | 339.1 | 8.3 | yes |
| 13 | urea, tetramethyl- | 367.6 | 19.4 | yes |
| 14 | glycerol | 367.6 | 103 | no |
| 15 | methanethioamide, N,N-dimethyl- | 384.4 | 4.3 | no |
| 16 | polyfluorinated compound with | 385.4 | 9.3 | no |
| | a maximum recorded weight of 397 | | | |
| 17 | benzoic acid ethyl ester | 462.5 | 13.9 | yes |
| 18 | cyclohexene, 3,5,5-trimethyl- | 466.2 | 25.9 | yes |
| 20 | naphthalene, 2-fluoro- | 473.3 | 36 | yes |
| 21 | naphthalene | 473.8 | 14.8 | yes |
| 22 | thiourea, tetramethyl- | 486.9 | 58 | yes |
| 23 | benzothiazole | 491.6 | 0.7 | yes |
| 25 | alkane | 559.9 | 26 | |
| 27 | dimethyl phthalate | 587 | 0.7 | no |
| 28 | alkane | 620.4 | 4.3 | |
| 29 | alkane | 626.6 | 5 | |
| 33 | alkane | 672.3 | 7.3 | |
| 34 | 3H-pyrazol-3-one, 2,4-dihydro- | 673.9 | 22 | yes |
| | 5-methyl-2-phenyl- | | | |
| 37 | alkane | 705.9 | 83 | |
| 39 | 5-ethoxy-3-methyl-1-phenylpyrazole- | 714.3 | 110 | yes |
| | carbonic acid | | | |
| 40 | diisobutyl phthalate | 759.5 | 23 | yes |
| 43 | alkane | 768 | 27 | |
| 47 | alkane | 824.7 | 25 | |
| 49 | alkane | 851.1 | 8.7 | |
| 50 | 1-benzyl-6-hydroxy-4-methyl-2(1H)- | 859.4 | 36 | yes |
| | oxo-3-pyridinecarbonitrile | | | |
| 52 | phenol, 2,2′-methylenebis[6-(1,1- | 889.6 | 7.7 | yes |
| | dimethylethyl)-4-methyl- | | | |
| 54 | alkane | 902.5 | 53 | |
| Total: | Toxic substance total concentration | | 507.1 | |

Thus, a PFOC-based gas transmission emulsion is created, the qualitative composition of which ensures the achievement of the technical result consisting in substantially reducing reactogenicity of the emulsion and making it possible to administer it to the subject.

1. A gas transmission emulsion that comprises a perfluororganic compound, a poloxamer and at least one additional excipient, characterized in that it comprises perfluoromethylcyclohexylpiperidine washed off of alcohol-soluble organic compounds with a 95% antiseptic medical solution and washed off of water-soluble organic compounds as a perfluororganic compound and comprises poloxamer-188 as a poloxamer and is intended for intravenous administration to the subject.

2. The gas transmission emulsion of claim 1, wherein the subject is a mammal.

3. The gas transmission emulsion of claim 1, wherein the additional excipient is sodium chloride.

4. The gas transmission emulsion of claim 1, wherein the concentration of perfluoromethylcyclohexylpiperidine washed off of alcohol-soluble and water-soluble organic compounds is 1-20 wt %.

5. The gas transmission emulsion of claim 1, wherein the concentration of the poloxamer-188 is 2-8 wt %.

6. The gas transmission emulsion of claim 3, wherein the concentration of sodium chloride is 0.1-0.9 wt %.
